(19) United States
(12) Patent Application Publication
(10) Pub. No.: US 2022/0310207 A1
(43) Pub. Date: Sep. 29, 2022

(54) SYSTEMS AND METHODS FOR GENETICALLY BASED BLOCKCHAIN

(71) Applicant: Robotic Research OpCo, LLC, Clarksburg, MD (US)

(72) Inventors: Alberto Daniel Lacaze, Potomac, MD (US); Kyle Smith, Montgomery Village, MD (US); Karl Nicholas Murphy, Cocoa Beach, FL (US)

(21) Appl. No.: 17/707,667

(22) Filed: Mar. 29, 2022

Related U.S. Application Data
(60) Provisional application No. 63/167,594, filed on Mar. 29, 2021.

(51) Int. Cl. G16B 40/00; G16B 50/30; G01N 33/00

(52) U.S. Cl. CPC G16B 40/00; G16B 50/30; G01N 33/0098

(57) ABSTRACT

Systems and methods for genetically based blockchains that encode blockchain entries into successive generations of organisms.

### CROSS-REFERENCE TO RELATED APPLICATIONS

This application claims benefit and priority under 35 U.S.C. § 119(e) to, and is a Non-provisional of, U.S. Provisional Patent Application No. 63/167,594 filed on Mar. 29, 2021 and titled “SYSTEMS AND METHODS FOR GENETICALLY BASED BLOCKCHAIN”, which is hereby incorporated by reference herein in its entirety.

### BACKGROUND

As blockchains gain in popularity, advantages and disadvantages of the mechanisms used by each blockchain are being discovered by the marketplace. As applied to currencies, the advantages are many, including the distributed ledger that protects the safety and integrity of the ownership of digital currencies, avoiding single points of failure present in fiat forgeries. Other advantages include the freedom/anonymity of exchange and the independence from centralized banks that allow the market to have greater power in determining the value of digital currencies.

Currently, many digital or “cyber”-currencies use proof of work as a method of acquiring coins and to protect the integrity of the ledger. One of the harshest complaints against the most popular blockchains and cybercurrencies is the amount of energy necessary to algorithmically mine coins. In other words, the “proof of work” requires computing large numbers of combinations that in turn spend energy in the process. The “proof of work” is inherent in the maintenance of the safety of the blockchain and therefore, these currencies/coins are inherently “dirty” from an environmental standpoint. Moreover, physical components like graphics cards and microchips are being expended in the calculation of “proof of work”, causing worldwide production and shortages of these items. To manufacture, these physical components require mining and other processes inherently “dirty” from an environmental standpoint. Lowering the number of manufactured graphics cards or microchips would be a positive for the environment.

The obvious downside of the current architecture is that a huge amount of energy is being spent to compute solutions to puzzles that have no practical use other than to maintain the safety of the blockchain and to demonstrate a “proof of work”. As these blockchains and cybercurrencies are becoming more popular, the environmental repercussions of such architectures become evident in their contribution to global warming, pollution, and general waste of energy. All of which are the byproducts of the state of the art in blockchain and cryptocurrency. Arguably this makes some of these cryptocurrencies more polluting than other (e.g., traditional) currencies based on mining of precious or rare metals.

### BRIEF DESCRIPTION OF THE DRAWINGS

The figures depict embodiments for purposes of illustration only. One skilled in the art will readily recognize from the following description that alternative embodiments of the systems and methods illustrated herein may be employed without departing from the principles described herein, wherein:

FIG. 1 is a block diagram of a genetically based blockchain system according to some embodiments;

FIG. 2 is a block diagram of a genetically based blockchain system according to some embodiments;

FIG. 3 is a flow diagram of a method according to some embodiments; and

FIG. 4 is a block diagram of an apparatus according to some embodiments.

### DETAILED DESCRIPTION

### I. Introduction

Embodiments of the invention described herein provide for an architecture where the “proof of work” (and/or “proof of stake”) and the distributed protection of a blockchain are created in an environmentally meaningful manner, solving many of the environmental-related problems of current cyber currency systems. In some embodiments, for example, the growth of biologic objects/biomass (e.g., trees, plants) may be utilized as the “proof of work” (and/or “proof of stake”) for a blockchain and/or the blockchain itself may be embedded into the generic material of the biomass. As opposed to the traditional architecture where special purpose silicon based electronics consume energy and therefore generate pollution and carbon emissions, according to some embodiments, the growth of the biomass, which consumes carbon dioxide (CO₂) and has other significant environmental positive outcomes, is used as a “proof of work” (and/or “proof of stake”). Similar to other types of cryptocurrency (e.g., bitcoin, Ethereum®), the replication of the blockchain may be distributed. Traditionally the blockchain is distributed to all the computers mining for coins around the world. According to some embodiments, the ledger may instead be embedded into the genetic material of a plant itself, providing the broad decentralized safety of the solution. It is expected that as a byproduct of the process, for example, wood (in the case that the biomass comprises a tree) could be utilized as a natural resource (e.g., a construction material, a food, and/or a fuel) accordingly preserving the integrity of the blockchain in a very physical and long-lasting way. In traditional cryptocurrency, energy spent testing wrong solutions to a puzzle is wasted energy. In accordance with some embodiments of the present invention, biomass that do not meet the criteria to become coins may retain an underlying value of the commodity (e.g., construction material, food, fuel, etc.).

In some embodiments, implementation of a genetically encoded blockchain may facilitate transformation of one of the most polluting industries into an environmental benefit. Embodiments may not only provide for blockchains that are environmentally sound, but may also create a serious method for worldwide carbon dioxide (CO₂) recapture and/or assist in reversing deforestation.

### II. Genetically Based Blockchain Systems

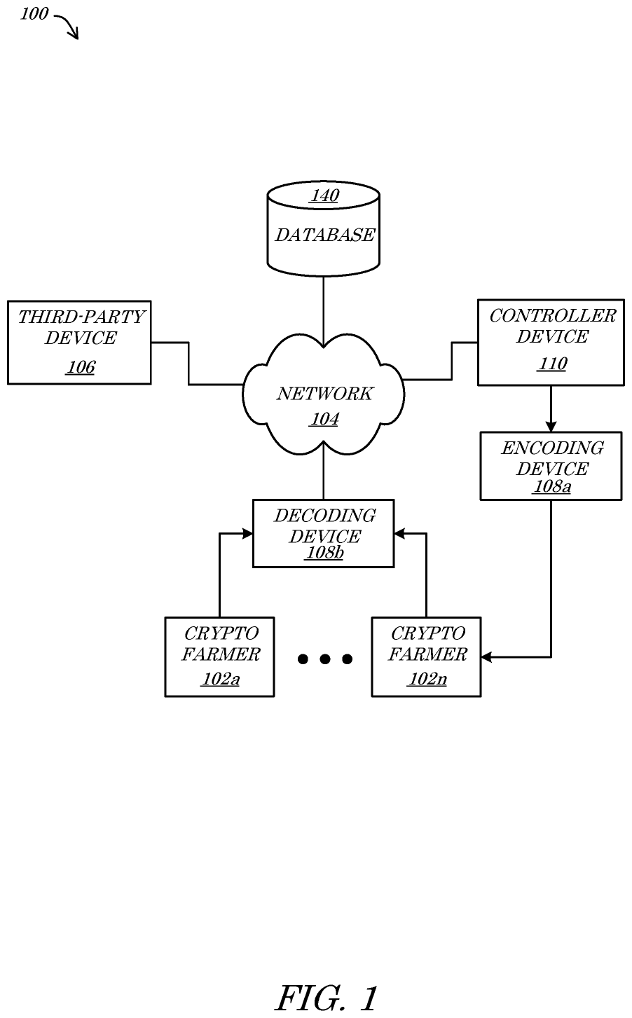

Embodiments described herein may be operable to make the “proof of work” (and/or “proof of stake”) portion of a blockchain create a positive environmental effect while still creating the advantages of safely maintaining a proper distributed ledger. According to some embodiments, genetically modified (e.g., by modified DeoxyriboNucleic Acid (DNA), RiboNucleic Acid (RNA), genes, chromosomes, etc.) biomass may be utilized to achieve this purpose. Referring initially to FIG. 1, for example, a block diagram of a genetically based blockchain system 100 according to some embodiments is shown. According to some embodiments, the genetically based blockchain system 100 may comprise a plurality of biomass and/or biologic object growing entities or “cyber farmers” or “crypto farmers” 102a-n. In some embodiments, a network 104 may communicatively couple the crypto farmers 102a-n with one or more of a third-party device 106, an encoding device 108a, a decoding device 108b, a controller device 110, and/or a database 140. In some embodiments, the third-party device 106 may comprise a user and/or transaction device associated with a new transaction for a blockchain and/or cryptocurrency and may communicate with the controller device 110 to initiate the transaction. According to some embodiments, the controller device 110 may instruct the encoding device 108a to encode data descriptive of the transaction into a genetic data pattern form and to transfer the genetic data pattern to a biologic object (e.g., plant cell; not separately shown). In some embodiments, the genetic data pattern may be stored in the database 140. In some embodiments, the genetically encoded biologic object may be provided to one or more of the crypto farmers 102a-n who then plant and/or grow biomass (also not separately shown) from the provided genetically encoded biologic object(s).

According to some embodiments, once the biomass reaches one or more predefined thresholds of progress, the decoding device 108b may extract the genetic data pattern from the biomass and, e.g., provide the extraction results to the controller device 110 (e.g., via the network 104). In some embodiments, the controller device 110 may analyze the decoding results to identify which (if any) of the crypto farmers 102a-n have achieve biomass growth results that qualify as “proof of work” and/or “proof of stake” (and/or proof of memory and/or data storage capacity) to establish a consensus to verify the transaction. In such a manner, for example, not only are vast amounts of energy that would otherwise be consumed by blockchain mining devices preserved, but the underlying storage medium—the biomass, may separately be utilized for an appropriate and likely valuable purpose (e.g., trees for lumber, food crops for food, fuel crops for fuel).

In some embodiments, the crypto farmers 102a-n may comprise any type or configuration of farming, horticulture, algaculture, fungiculture, silviculture, and/or aquaculture systems and/or devices that are or become known or practicable. The crypto farmers 102a-n may comprise, for example, various fields, equipment, buildings, and/or resources such as water, nutrients, etc. that are operable to grow at least one unit of desired biomass to a meet a predetermined threshold criterion.

The network 104 may, according to some embodiments, comprise a Local Area Network (LAN; wireless and/or wired), cellular telephone, Bluetooth®, Near Field Communication (NFC), and/or Radio Frequency (RF) network with communication links between the controller device 110, the crypto farmers 102a-n, the third-party device 106, and/or the database 140. In some embodiments, the network 104 may comprise direct communication links between any or all of the components 102a-n, 106, 108a-b, 110, 140 of the system 100. The crypto farmers 102a-n may, for example, be directly interfaced or connected to one or more of the controller device 110 and/or the third-party device 106 via one or more wires, cables, wireless links, and/or other network components, such network components (e.g., communication links) comprising portions of the network 104. In some embodiments, the network 104 may comprise one or many other links or network components other than those depicted in FIG. 1. The controller device 110 may, for example, be connected to one or more of the crypto farmers 102a-n via various cell towers, routers, repeaters, ports, switches, and/or other network components that comprise the Internet and/or a cellular telephone (and/or Public Switched Telephone Network (PSTN)) network, and which comprise portions of the network 104.

While the network 104 is depicted in FIG. 1 as a single object, the network 104 may comprise any number, type, and/or configuration of networks that is or becomes known or practicable. According to some embodiments, the network 104 may comprise a conglomeration of different sub-networks and/or network components interconnected, directly or indirectly, by the components 102a-n, 106, 108a-b, 110, 140 of the system 100. The network 104 may comprise one or more cellular telephone networks with communication links between the decoding device 108b and the controller device 110, for example, and/or may comprise an NFC or other short-range wireless communication path, with communication links between various crypto farmers 102a-n, for example.

According to some embodiments, the third-party device 106 may comprise any type or configuration of a computerized processing device, such as a PC, laptop computer, computer server, database system, and/or other electronic device, devices, or any combination thereof. In some embodiments, the third-party device 106 may be owned and/or operated by a third-party (i.e., an entity different than any entity owning and/or operating either the crypto farmers 102a-n or the controller device 110; such as a smart contract and/or cryptocurrency transaction user). The third-party device 106 may, for example, comprise a consumer or financial institution device that requests a transaction be added to a blockchain associated with the system 100.

In some embodiments, the controller device 110 may comprise an electronic and/or computerized controller device, such as a computer server and/or server cluster communicatively coupled to interface with the crypto farmers 102a-n, the encoding/decoding devices 108a-b, and/or the third-party device 106 (directly and/or indirectly). The controller device 110 may, for example, comprise one or more PowerEdge™ M910 blade servers manufactured by Dell®, Inc. of Round Rock, Tex., which may include one or more Eight-Core Intel® Xeon® 7500 Series electronic processing devices. According to some embodiments, the controller device 110 may be located remotely from one or more of the crypto farmers 102a-n and/or the third-party device 106. The controller device 110 may also or alternatively comprise a plurality of electronic processing devices located at one or more various sites and/or locations (e.g., a distributed computing and/or processing network).

According to some embodiments, the controller device 110 may store and/or execute specially programmed instructions (not separately shown in FIG. 1) to operate in accordance with embodiments described herein. The controller device 110 may, for example, execute one or more programs, modules, and/or routines that facilitate the management of a genetically based blockchain, as described herein. According to some embodiments, the controller device 110 may execute stored instructions, logic, and/or software modules to (i) identify transaction data, (ii) encode transaction data into genetic data, (iii) transfer the genetic data to a biologic host/object, (iv) disseminate the biologic host/object to the crypto farmers 102a-n for propagation/growth, (v) measure, analyze, and/or decode biomass grown by one or more of the crypto farmers 102a-n, and/or (vi) validate one or more blockchain transactions based on the measured, analyzed, and/or decoded biomass.

In some embodiments, the crypto farmers 102a-n, the third-party device 106, and/or the controller device 110 may be in communication with and/or comprise the database 140. The database 140 may comprise, for example, various databases and/or data storage mediums that may store, for example, genetic data, cryptographic keys and/or data, login and/or identity credentials, and/or instructions that cause various devices (e.g., the controller device 110, the third-party device 106, and/or the encoding/decoding devices 108a-b) to operate in accordance with embodiments described herein.

In some embodiments, the memory device 140 may comprise any type, configuration, and/or quantity of data storage devices that are or become known or practicable. The memory device 140 may, for example, comprise an array of optical and/or solid-state hard drives configured to store genetic data, credentialing instructions and/or keys, and/or various operating instructions, drivers, etc. In some embodiments, the memory device 140 may comprise a solid-state and/or non-volatile memory card (e.g., a Secure Digital (SD) card, such as an SD Standard-Capacity (SDSC), an SD High-Capacity (SDHC), and/or an SD eXtended-Capacity (SDXC) and any various practicable form-factors, such as original, mini, and micro sizes, such as are available from Western Digital Corporation of San Jose, Calif. While the memory device 140 is depicted as a stand-alone component, the memory device 140 may comprise multiple components. In some embodiments, a multi-component memory device 140 may be distributed across various devices and/or may comprise remotely dispersed components. Any or all of the crypto farmers 102a-n, the third-party device 106, and/or the controller device 110 may comprise the memory device 140 or a portion thereof, for example.

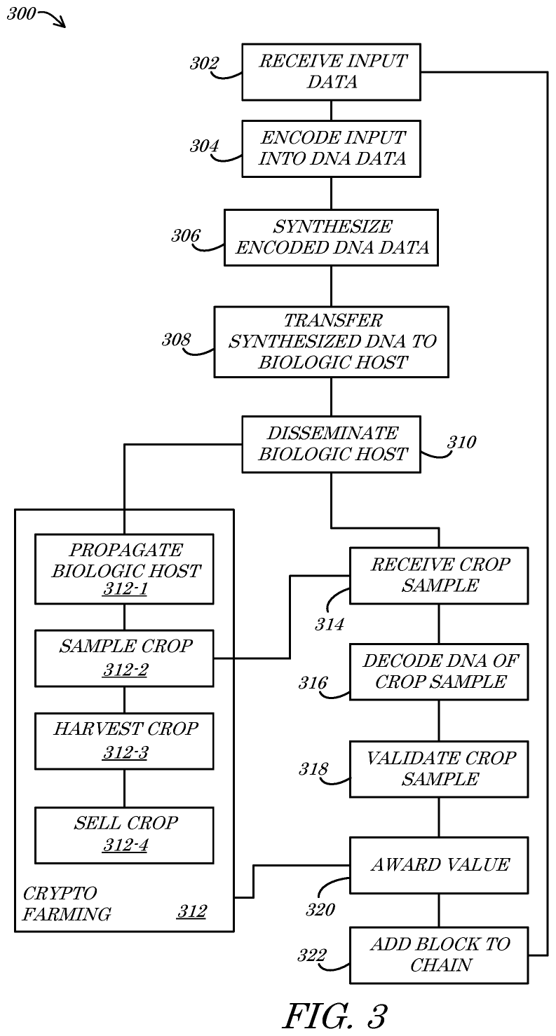

Fewer or more components 102a-n, 104, 106, 108a-b, 110, 140 and/or various configurations of the depicted components 102a-n, 104, 106, 108a-b, 110, 140 may be included in the genetically based blockchain system 100 without deviating from the scope of embodiments described herein. In some embodiments, the components 102a-n, 104, 106, 108a-b, 110, 140 may be similar in configuration and/or functionality to similarly named and/or numbered components as described herein. In some embodiments, the genetically based blockchain system 100 (and/or portion thereof) may comprise genetically based blockchain system and/or platform programmed and/or otherwise configured to execute (e.g., via the controller device 110), conduct, and/or facilitate methods described herein such as the method 300 of FIG. 3 herein, or portions thereof.

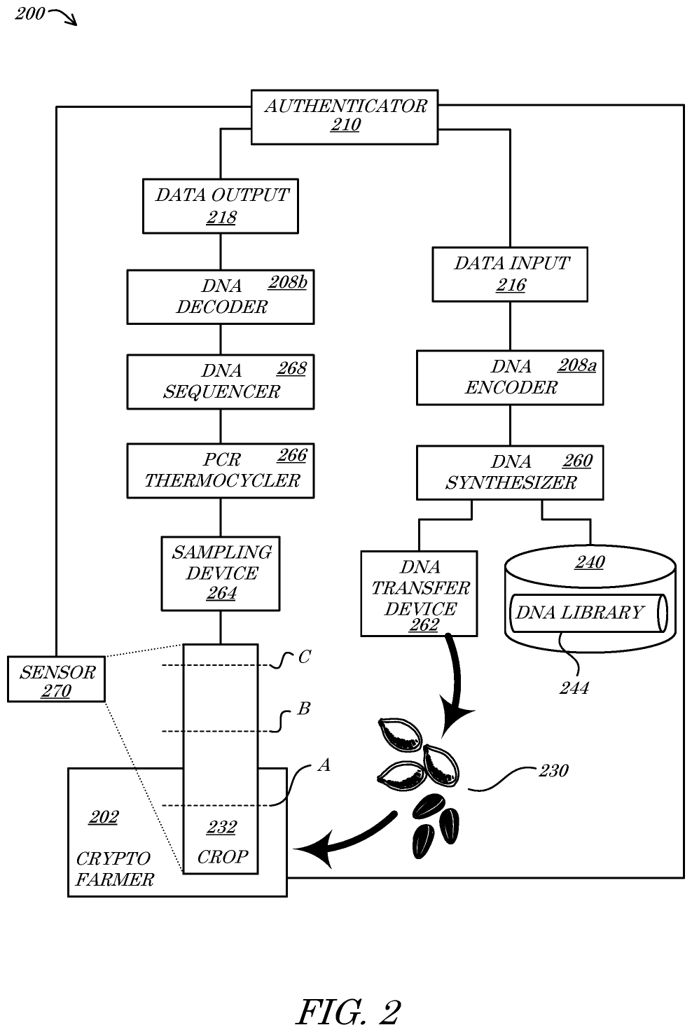

Turning to FIG. 2, a block diagram of a genetically based blockchain system 200 according to some embodiments is shown. In some embodiments, the genetically based blockchain system 200 may comprise a crypto farmer 202 and/or a DNA encoder 208a, a DNA decoder 208b, and/or an authenticator 210. The authenticator 210 may comprise a blockchain fabric “orderer” and/or ordering service or management device, for example, that utilizes the DNA encoder 208a and a DNA decoder 208b to translate digital transaction data to (e.g., data input 216) and from (e.g., data output 218) genetic data that is propagated and/or stored by actual work of the crypto farmer 202. In some embodiments, genetically modified seeds (and/or grafts) 230 may be grown by the crypto farmer 202, e.g., to produce an amount of biomass or a “crop” 232. This resulting genetically modified crop/biomass 232 (e.g., trees, algae, bacteria, food crops) may, in some embodiments, contain a copy of a distributed ledger in their cells. As the crop 232 grows, there may be a set of circumstances and/or a trigger that may determine the threshold necessary to achieve a coin (or other unit of currency), a transaction validation, and/or other milestone and therefore constitute a new entry into the blockchain ledger (e.g., a stored modified genetic code). If the triggering and/or achievement conditions are met, the new entry is spliced into the genetic material of the next generation of seeds/offspring for that (or other crops) using Clustered Regularly Interspaced Short Palindromic Repeats (CRISPR) and/or CRISPR-associated protein 9 (CRISPR-Cas9), Agrobacterium tumefaciens, a particle gun, and/or other known methods of gene splicing and/or editing.

The genetically based blockchain system 200 may comprise, for example, a database 240 storing a DNA library 244 that is accessible to a DNA synthesizer 260. In some embodiments, any transaction data identified by the authenticator 210 may be mapped to a combination of one of the four nitrogen-containing nucleobases cytosine “C”, guanine “G”, adenine “A”, and thymine “T” by the DNA encoder 208a. According to some embodiments, the DNA encoder 208a may comprise a computer program and/or model that operates in accordance with a set of predetermined mapping rules (e.g., binary to DNA) such as in accordance with the Adaptive DNA Storage Codex (ADS Codex) developed by the Los Alamos National Laboratories in Los Alamos, N.Mex. In some embodiments, the encoded data (e.g., genetic code specific to the transaction) may be incorporated into a pre-existing genetic code, e.g., stored in the DNA library 244, by a DNA synthesizer 260 such as a Mermade™ 192X Synthesizer available from Biosearch™ Technologies of LGC Limited, Middlesex, UK. The genetic code may then, for example, be incorporated into a biologic object such as a seed or graft via a DNA transfer device 262 such as a particle gun, Agrobacterium tumefaciens device, CRISPR-Cas9 device, etc.

According to some embodiments, the ledger (e.g., modified genetic code) may be maintained through Cisgenic or Transgenic processes. It is common practice with Genetically Modified Organism (GMO) seeds that farmers sign a contract not to plant the seeds sown by their crops. In some embodiments, restrictions on seeds (not separately shown) from the biomass 232 may not be necessary, as newly harvested seeds will lack new blockchain entries and therefore would not help to contribute to the mining/farming process although, they may still be useful for the environmental causes and/or as a traditional commodity. According to some embodiments, a GMO seed producer may entice and/or increase sales of new seed batches (e.g., the seeds 230) as old seed batches would have an obsolete copy of the ledger and therefore no longer be part of future blockchain entries.

In some embodiments, once the crop 232 reaches a predetermined level, threshold, and/or milestone (e.g., “A”, “B”, or “C”), a sampling device 264 may be utilized to extract genetic material from the crop 232. The sampling device 264 may comprise, for example, one or more manual and/or automated mechanical devices operable to extract a portion of the crop 232 for analysis (e.g., a harvester, scythe, clipper, trimmer, grafting tool, de-seeding device). According to some embodiments, a Polymerase Chain Reaction (PCR) thermocycler 266 may be utilized to amplify segments of the genetic code of the sampled crop 232. The PCR thermocycler 266 may comprise, for example, a QIAamplifier™ 96 available from QIAGEN of Hilden, Germany. In some embodiments, the amplified DNA/genetic code (or unamplified in the case that the PCR thermocycler 266 is not utilized) may be extracted by a DNA sequencer 268. The DNA sequencer 268 may comprise, for example, an Applied Biosystems™ 3500 Dx Series Genetic Analyzer available from Applied Biosystems, of the Life Technologies brand of Thermo Fisher Scientific Corporation of Waltham, Mass. According to some embodiments, the genetic code/DNA sequence of the crop 232 obtained by the DNA sequencer 268 may be processed by the DNA decoder 208b (e.g., that may operate by applying the same (and/or reverse) codex and/or algorithm utilized to encode the original genetic data via the DNA encoder 208a) to obtain blockchain data (e.g., retrieved from the DNA of the crop 232) such as the data output 218. In some embodiments, the authenticator 210 may validate the data output 218 and may pass the data output 218 and/or other data as new data input 216 to produce a new generation of updated blockchain genetic storage.

According to some embodiments, the genetically based blockchain system 200 may comprise a sensor 270 coupled to measure and/o take readings descriptive of the crop 232. The sensor 232 may comprise, for example, a LADAR, temperature, IR, mass, color, moisture, gas (e.g., oxygen and/or CO₂), and/or other sensor coupled to gather data descriptive of the crop 232 and pass the data to the authenticator 210. The authenticator 210 may analyze the data, for example, to determine if/when the crop 232 reaches one or more of the predetermined levels, thresholds, and/or milestones “A”, “B”, or “C”. In some embodiments, one or more of the milestones “A”, “B”, or “C” (e.g., a predetermined height, mass, number of branches, leaves, fruit, etc.) may be utilized as a trigger for the sampling device 264 to initiate genetic blockchain code extraction. According to some embodiments, whether the crop 232 qualifies as a validated blockchain entry and/or work product may be based upon whether (and/or when—e.g., how long it takes) the crop 232 to reach one of more of the milestones “A”, “B”, or “C”. In some embodiments, whether utilized as proof of work, stake, memory/capacity, and/or another blockchain-based performance metric, in the case that the sensor 270 acquires data that the authenticator 210 determines qualifies the crop 232 for blockchain purposes, the crypto farmer 202 may awarded a transaction fee and/or a new unit of genetically based crypto currency (e.g., based on the crop 232).

Fewer or more components 202, 208a-b, 210, 216, 218, 230, 232, 240, 244, 260, 262, 264, 266, 268, 270 and/or various configurations of the depicted components 202, 208a-b, 210, 216, 218, 230, 232, 240, 244, 260, 262, 264, 266, 268, 270 may be included in the genetically based blockchain system 200 without deviating from the scope of embodiments described herein. In some embodiments, the components 202, 208a-b, 210, 216, 218, 230, 232, 240, 244, 260, 262, 264, 266, 268, 270 may be similar in configuration and/or functionality to similarly named and/or numbered components as described herein. In some embodiments, the genetically based blockchain system 200 (and/or portion thereof) may comprise genetically based blockchain system and/or platform programmed and/or otherwise configured to execute (e.g., via the authenticator 210), conduct, and/or facilitate methods described herein such as the method 300 of FIG. 3 herein, or portions thereof.

### III. Genetically Based Blockchain Methods

Turning to FIG. 3, a flowchart of a method 300 according to some embodiments is shown. In some embodiments, a method 300 may comprise a method of implementing a genetically based blockchain that may comprise one or more of the following actions: (i) receiving input data (e.g., transaction data), at 302; (ii) encoding the input data into DNA data, at 304; (iii) synthesizing the encoded DNA data, at 306; (iv) transferring the synthesized DNA data to a biologic host, at 308—e.g., blockchain-enhanced (i.e., containing a copy of the distributed ledger) seeds/saplings/grafts/tubers/etc. (e.g., growable media) are prepared by gene splicing and/or editing; (v) disseminating the biologic host, at 310—e.g., a genetically based blockchain “miner” or crypto farmer purchases one or more blockchain-enhanced (i.e., containing the ledger) seeds and/or other growable media; (vi) the crypto farmer conducts “crypto farming”, at 312, by (a) propagating the biologic host, at 312-1, e.g., the farmer grows a unit of biomass (e.g., a tree) utilizing the blockchain-enhanced growable media, (b) sampling the crop, at 312-2, (c) harvesting the crop, at 312-3, and/or selling the crop, at 312-4 (e.g., the harvested biomass is sold in the market for uses unrelated to blockchains or cryptocurrency (e.g., food, construction, crafts)); (vii) receiving the crop sample (and/or harvest) from the farmer, at 314—e.g., to determine whether a growth goal and/or a harvesting goal is reached; (viii) decoding the DNA of the crop sample, at 316; (ix) validating the crop sample, at 318, e.g., the harvest is inspected for criteria related to suitability to create a new block for the blockchain; (x) awarding value to the farmer, at 320; (xi) adding the new block to the block chain, at 322, e.g., by generating new seeds (and/or other growable media) that add the latest blockchain entries into the genetic code of the biomass (e.g., the “genetic ledger”); and/or (xii) the process is repeated.

According to some embodiments, various triggers and/or criteria may be evaluated to determine whether a harvest has achieved a currency value, event, and/or status. In the case that the criteria/trigger is determined to have been met, information indicative of the harvest may comprise and/or define a new entry for the blockchain. In some embodiments, triggers and/or criteria may include, but are not limited to: (i) the sample/harvested plant has reached a certain level of maturity; (ii) the sample/harvested plant's DNA contains a ledger that shows the correct sequence and is historically valid with respect to other ledgers; (iii) the sample/harvested plant comprises a certain number of mutations that match a specific criteria used for validating “proof of work” for creation of a new blockchain entry; (iv) the sample/harvested plant comprises crossover that match a specific criteria used for validating “proof of work” for creation of a new blockchain entry; and/or (v) the size, weight, color, texture, and/or other physical signature of the sample/harvested plant meets one or more predetermined criteria for, e.g., “proof of stake” or “proof of memory”.

The match criteria can be a random signature that is independently created or is automatically created by a combination of data included in the ledger. Similar to bitcoin and other currencies, the criteria can be a random nonce that goes into a block in the blockchain and makes the block have a hash that starts with a certain number of zeros (0's). In some embodiments, the nonces and the hash may be based on a base arithmetic inherent to gene expressions. According to some embodiments, and similar to how traditional cybercurrencies operate, the number of zeros (0's) may be utilized to adjust the difficulty of the solutions. Random mutations and/or crossover are natural methods for the plants to achieve these matches. Simpler matches may be utilized in such cases as the mutations on the plants are significantly more sporadic than the many billions of combinations used for cybercurrencies. However, it is possible to accelerate the mutation process in these plants and selectively allow mutation only in certain areas of the DNA chains, therefore, there are many options for the particular criteria being used. If the biomass being used is algae, computationally expensive hashes or other tests can be used as the criteria. In some embodiments, the criteria can be based on gene expressions that only manifest at a certain age of the plant, and therefore, further guarantee the “proof of work” process.

In some embodiments, public entities may purchase these new environmentally conscious coins/currency units to increase the value of the coins/units and therefore reward crypto farmer for their work, and at the same time, increase reforestation goals. Crops can be rotated based on need or based on the climate or geographical parameters. Multi-species hashes can be used where the crypto farmers may be required to have matches across multiple plants to get a coin/unit to increase forestation goals in certain areas. To aid in the whole globe being involved, different types of biomass can be utilized so that the natural biome of an area is not negatively impacted. One advantage of some embodiments is that it can provide an incentive to grow plants that do not provide a significant cash commodity but still produce significant environmental advantages. For example, biomass that provides habitat for endangered species, but take a long time to grow or are not good cash crops, may be more attractive to grow due to the genetically based blockchain value thereof.

According to some embodiments, trees (and/or other organisms) planted in accordance with the genetically based blockchain could be utilized to create a new blockchain-verified carbon credit. The crypto farmer could sell the carbon credit to a polluter. The blockchain could be managed centrally by a validation authority. The carbon credit may be created and buying a carbon “coin” would give you “permission” to pollute a certain amount. In some embodiments, the government and/or a third party may verify the carbon credit to prove the work to get added into the blockchain.

### IV. Genetically Based Blockchain Apparatus

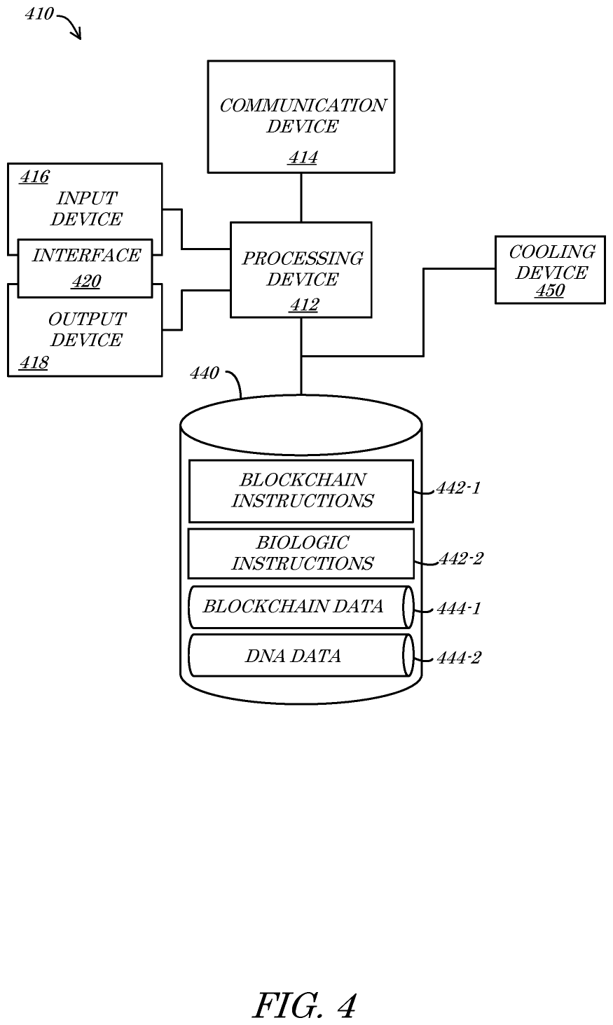

Turning to FIG. 4, a block diagram of an apparatus 410 according to some embodiments is shown. In some embodiments, the apparatus 410 may be similar in configuration and/or functionality to one or more of the controller device 110 and/or the authenticator 210 of FIG. 1 and/or FIG. 2 herein. The apparatus 410 may, for example, execute, process, facilitate, and/or otherwise be associated with the method 300 of FIG. 3 herein, and/or portions thereof. In some embodiments, the apparatus 410 may comprise a processing device 412, a communication device 414, an input device 416, an output device 418, an interface 420, a memory device 440 (storing various programs and/or instructions 442 and data 444), and/or a cooling device 450. According to some embodiments, any or all of the components 412, 414, 416, 418, 420, 440, 442, 444, 450 of the apparatus 410 may be similar in configuration and/or functionality to any similarly named and/or numbered components described herein. Fewer or more components 412, 414, 416, 418, 420, 440, 442, 444, 450 and/or various configurations of the components 412, 414, 416, 418, 420, 440, 442, 444, 450 may be included in the apparatus 410 without deviating from the scope of embodiments described herein.

According to some embodiments, the processor 412 may be or include any type, quantity, and/or configuration of processor that is or becomes known. The processor 412 may comprise, for example, an Intel® IXP 2800 network processor or an Intel® XEON™ Processor coupled with an Intel® E7501 chipset. In some embodiments, the processor 412 may comprise multiple inter-connected processors, microprocessors, and/or micro-engines. According to some embodiments, the processor 412 (and/or the apparatus 410 and/or other components thereof) may be supplied power via a power supply (not shown) such as a battery, an Alternating Current (AC) source, a Direct Current (DC) source, an AC/DC adapter, solar cells, and/or an inertial generator. In the case that the apparatus 410 comprises a server, such as a blade server, necessary power may be supplied via a standard AC outlet, power strip, surge protector, and/or Uninterruptible Power Supply (UPS) device.

In some embodiments, the communication device 414 may comprise any type or configuration of communication device that is or becomes known or practicable. The communication device 414 may, for example, comprise a Network Interface Card (NIC), a telephonic device, a cellular network device, a router, a hub, a modem, and/or a communications port or cable. In some embodiments, the communication device 414 may be coupled to receive transaction input data, e.g., from a consumer device (not shown in FIG. 4). The communication device 414 may, for example, comprise a BLE and/or RF receiver device and/or a camera or other imaging device that acquires data descriptive of a genetically modified crop (not separately depicted in FIG. 4) and/or a transmitter device that provides the data to a remote server and/or server or communications layer (also not separately shown in FIG. 4). According to some embodiments, the communication device 414 may also or alternatively be coupled to the processor 412. In some embodiments, the communication device 414 may comprise an IR, RF, Bluetooth™, Near-Field Communication (NFC), and/or Wi-Fi® network device coupled to facilitate communications between the processor 412 and another device (such as a remote user device, not separately shown in FIG. 4).

In some embodiments, the input device 416 and/or the output device 418 are communicatively coupled to the processor 412 (e.g., via wired and/or wireless connections and/or pathways) and they may generally comprise any types or configurations of input and output components and/or devices that are or become known, respectively. The input device 416 may comprise, for example, a keyboard that allows an operator of the apparatus 410 to interface with the apparatus 410. In some embodiments, the input device 416 may comprise a sensor, such as a camera, sound, light, weight, sugar content, moisture content, and/or other sensor, configured to measure crop values and report measured values via signals to the apparatus 410 and/or the processor 412. The output device 418 may, according to some embodiments, comprise a display screen and/or other practicable output component and/or device. The output device 418 may, for example, provide an interface (such as the interface 420) via which functionality for genetically based blockchain transactions is provided to a user (e.g., via a website and/or mobile application). According to some embodiments, the input device 416 and/or the output device 418 may comprise and/or be embodied in a single device, such as a touch-screen monitor.

The memory device 440 may comprise any appropriate information storage device that is or becomes known or available, including, but not limited to, units and/or combinations of magnetic storage devices (e.g., a hard disk drive), optical storage devices, and/or semiconductor memory devices such as RAM devices, Read Only Memory (ROM) devices, Single Data Rate Random Access Memory (SDR-RAM), Double Data Rate Random Access Memory (DDR-RAM), and/or Programmable Read Only Memory (PROM). The memory device 440 may, according to some embodiments, store one or more of blockchain instructions 442-1, biologic instructions 442-2, blockchain data 444-1, and/or DNA data 444-2. In some embodiments, the blockchain instructions 442-1, biologic instructions 442-2, blockchain data 444-1, and/or DNA data 444-2 may be utilized by the processor 412 to provide output information via the output device 418 and/or the communication device 414.

According to some embodiments, the blockchain instructions 442-1 may be operable to cause the processor 412 to process the blockchain data 444-1 and/or DNA data 444-2 in accordance with embodiments as described herein. Blockchain data 444-1 and/or DNA data 444-2 received via the input device 416 and/or the communication device 414 may, for example, be analyzed, sorted, filtered, decoded, decompressed, ranked, scored, plotted, and/or otherwise processed by the processor 412 in accordance with the blockchain instructions 442-1. In some embodiments, blockchain data 444-1 and/or DNA data 444-2 may be fed by the processor 412 through one or more mathematical and/or statistical formulas and/or models in accordance with the blockchain instructions 442-1 to generate, verify, and/or store genetically based blockchain data blocks, as described herein.

In some embodiments, the biologic instructions 442-2 may be operable to cause the processor 412 to process the blockchain data 444-1 and/or DNA data 444-2 in accordance with embodiments as described herein. Blockchain data 444-1 and/or DNA data 444-2 received via the input device 416 and/or the communication device 414 may, for example, be analyzed, sorted, filtered, decoded, decompressed, ranked, scored, plotted, and/or otherwise processed by the processor 412 in accordance with the biologic instructions 442-2. In some embodiments, blockchain data 444-1 and/or DNA data 444-2 may be fed by the processor 412 through one or more mathematical and/or statistical formulas and/or models in accordance with the biologic instructions 442-2 to encode, decode, sample, and/or create genetically modified biologic objects (e.g., seeds, grafts, tubers, etc.), as described herein.

According to some embodiments, the apparatus 410 may comprise the cooling device 450. According to some embodiments, the cooling device 450 may be coupled (physically, thermally, and/or electrically) to the processor 412 and/or to the memory device 440. The cooling device 450 may, for example, comprise a fan, heat sink, heat pipe, radiator, cold plate, and/or other cooling component or device or combinations thereof, configured to remove heat from portions or components of the apparatus 410.

Any or all of the exemplary instructions and data types described herein and other practicable types of data may be stored in any number, type, and/or configuration of memory devices that is or becomes known. The memory device 440 may, for example, comprise one or more data tables or files, databases, table spaces, registers, and/or other storage structures. In some embodiments, multiple databases and/or storage structures (and/or multiple memory devices 440) may be utilized to store information associated with the apparatus 410. According to some embodiments, the memory device 440 may be incorporated into and/or otherwise coupled to the apparatus 410 (e.g., as shown) or may simply be accessible to the apparatus 410 (e.g., externally located and/or situated).

### V. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Neither the Title (set forth at the beginning of the first page of this patent application) nor the Abstract (set forth at the end of this patent application) is to be taken as limiting in any way as the scope of the disclosed invention(s). Headings of sections provided in this patent application are for convenience only, and are not to be taken as limiting the disclosure in any way.

All definitions, as defined and used herein, should be understood to control over dictionary definitions, definitions in documents incorporated by reference, and/or ordinary meanings of the defined terms. The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

The indefinite articles “a” and “an,” as used herein in the specification and in the claims, unless clearly indicated to the contrary, should be understood to mean “at least one” or “one or more”.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a FDA” does not imply that any or all of the three items of that list are mutually exclusive and does not imply that any or all of the three items of that list are comprehensive of any category.

Some embodiments described herein are associated with a “user device” or a “network device”. As used herein, the terms “user device” and “network device” may be used interchangeably and may generally refer to any device that can communicate via a network. Examples of user or network devices include a PC, a workstation, a server, a printer, a scanner, a facsimile machine, a copier, a Personal Digital Assistant (PDA), a storage device (e.g., a disk drive), a hub, a router, a switch, and a modem, a video game console, or a wireless phone. User and network devices may comprise one or more communication or network components. As used herein, a “user” may generally refer to any individual and/or entity that operates a user device. Users may comprise, for example, customers, consumers, product underwriters, product distributors, customer service representatives, agents, brokers, etc.

As used herein, the term “network component” may refer to a user or network device, or a component, piece, portion, or combination of user or network devices. Examples of network components may include a Static Random Access Memory (SRAM) device or module, a network processor, and a network communication path, connection, port, or cable.

In addition, some embodiments are associated with a “network” or a “communication network”. As used herein, the terms “network” and “communication network” may be used interchangeably and may refer to any object, entity, component, device, and/or any combination thereof that permits, facilitates, and/or otherwise contributes to or is associated with the transmission of messages, packets, signals, and/or other forms of information between and/or within one or more network devices. Networks may be or include a plurality of interconnected network devices. In some embodiments, networks may be hard-wired, wireless, virtual, neural, and/or any other configuration of type that is or becomes known. Communication networks may include, for example, one or more networks configured to operate in accordance with the Fast Ethernet LAN transmission standard 802.3-2002® published by the Institute of Electrical and Electronics Engineers (IEEE). In some embodiments, a network may include one or more wired and/or wireless networks operated in accordance with any communication standard or protocol that is or becomes known or practicable.

As used herein, the terms “information” and “data” may be used interchangeably and may refer to any data, text, voice, video, image, message, bit, packet, pulse, tone, waveform, and/or other type or configuration of signal and/or information. Information may comprise information packets transmitted, for example, in accordance with the Internet Protocol Version 6 (IPv6) standard as defined by “Internet Protocol Version 6 (IPv6) Specification” RFC 1883, published by the Internet Engineering Task Force (IETF), Network Working Group, S. Deering et al. (December 1995). Information may, according to some embodiments, be compressed, encoded, encrypted, and/or otherwise packaged or manipulated in accordance with any method that is or becomes known or practicable.

In addition, some embodiments described herein are associated with an “indication”. As used herein, the term “indication” may be used to refer to any indicia and/or other information indicative of or associated with a subject, item, entity, and/or other object and/or idea. As used herein, the phrases “information indicative of” and “indicia” may be used to refer to any information that represents, describes, and/or is otherwise associated with a related entity, subject, or object. Indicia of information may include, for example, a code, a reference, a link, a signal, an identifier, and/or any combination thereof and/or any other informative representation associated with the information. In some embodiments, indicia of information (or indicative of the information) may be or include the information itself and/or any portion or component of the information. In some embodiments, an indication may include a request, a solicitation, a broadcast, and/or any other form of information gathering and/or dissemination.

As utilized herein, the terms “program” or “computer program” may refer to one or more algorithms formatted for execution by a computer. The term “module” or “software module” refers to any number of algorithms and/or programs that are written to achieve a particular output and/or output goal—e.g., a ‘login credentialing’ module (or program) may provide functionality for permitting a user to login to a computer software and/or hardware resource and/or a ‘shipping’ module (or program) may be programmed to electronically initiate a shipment of an object via a known and/or available shipping company and/or service (e.g., FedEX®). The terms “engine” or “software engine” refer to any combination of software modules and/or algorithms that operate upon one or more inputs to define one or more outputs in an ongoing, cyclical, repetitive, and/or loop fashion. Data transformation scripts and/or algorithms that query data from a data source, transform the data, and load the transformed data into a target data repository may be termed ‘data transformation engines’, for example, as they repetitively operate in an iterative manner upon each row of data to produce the desired results.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

“Determining” something can be performed in a variety of manners and therefore the term “determining” (and like terms) includes calculating, computing, deriving, looking up (e.g., in a table, database or data structure), ascertaining and the like.

It will be readily apparent that the various methods and algorithms described herein may be implemented by, e.g., appropriately and/or specially-programmed computers and/or computing devices. Typically a processor (e.g., one or more microprocessors) will receive instructions from a memory or like device, and execute those instructions, thereby performing one or more processes defined by those instructions. Further, programs that implement such methods and algorithms may be stored and transmitted using a variety of media (e.g., computer readable media) in a number of manners. In some embodiments, hard-wired circuitry or custom hardware may be used in place of, or in combination with, software instructions for implementation of the processes of various embodiments. Thus, embodiments are not limited to any specific combination of hardware and software

A “processor” generally means any one or more microprocessors, CPU devices, computing devices, microcontrollers, digital signal processors, or like devices, as further described herein.

The term “computer-readable medium” refers to any medium that participates in providing data (e.g., instructions or other information) that may be read by a computer, a processor or a like device. Such a medium may take many forms, including but not limited to, non-volatile media, volatile media, and transmission media. Non-volatile media include, for example, optical or magnetic disks and other persistent memory. Volatile media include DRAM, which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during RF and IR data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave, or any other medium from which a computer can read.

The term “computer-readable memory” may generally refer to a subset and/or class of computer-readable medium that does not include transmission media such as waveforms, carrier waves, electromagnetic emissions, etc. Computer-readable memory may typically include physical media upon which data (e.g., instructions or other information) are stored, such as optical or magnetic disks and other persistent memory, DRAM, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, computer hard drives, backup tapes, Universal Serial Bus (USB) memory devices, and the like.

Various forms of computer readable media may be involved in carrying data, including sequences of instructions, to a processor. For example, sequences of instruction (i) may be delivered from RAM to a processor, (ii) may be carried over a wireless transmission medium, and/or (iii) may be formatted according to numerous formats, standards or protocols, such as Bluetooth™, TDMA, CDMA, 3G.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models and/or distributed databases) could be used to store and manipulate the data types described herein. Likewise, object methods or behaviors of a database can be used to implement various processes, such as the described herein. In addition, the databases may, in a known manner, be stored locally or remotely from a device that accesses data in such a database.

The present invention can be configured to work in a network environment including a computer that is in communication, via a communications network, with one or more devices. The computer may communicate with the devices directly or indirectly, via a wired or wireless medium such as the Internet, LAN, WAN or Ethernet, Token Ring, or via any appropriate communications means or combination of communications means. Each of the devices may comprise computers, such as those based on the Intel® Pentium® or Centrino™ processor, that are adapted to communicate with the computer. Any number and type of machines may be in communication with the computer.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

What is claimed is:

1. An environmentally conscious genetically based blockchain method, comprising:
receiving digital transaction input;
encoding the digital transaction input into genetic data;
synthesizing the encoded genetic data;
transferring the synthesized encoded genetic data to a biologic host;
disseminating the biologic host to a plurality of crypto farmers;
receiving, after the disseminating, a crop sample from at least one of the plurality of crypto farmers;
decoding at least a portion of the DNA of the crop sample;
validating the decoded DNA of the crop sample; and
awarding, in response to the validating, the at least one of the plurality of crypto farmers a unit of value.

2. The method of claim 1, further comprising:
encoding data descriptive of the validation of the crop sample into new genetic data;
synthesizing the new encoded genetic data;
transferring the synthesized new encoded genetic data to a new biologic host; and
disseminating the new biologic host to the plurality of crypto farmers.

3. The method of claim 1, further comprising:
encoding data descriptive of the awarding of the unit of value into new genetic data;
synthesizing the new encoded genetic data;
transferring the synthesized new encoded genetic data to a new biologic host; and
disseminating the new biologic host to the plurality of crypto farmers.

4. The method of claim 1, wherein the validating, comprises:
comparing the decoded DNA of the crop sample to reference data stored in a database.

5. The method of claim 1, wherein the validating, comprises:
measuring at least one of a height and a mass of the crop sample;
comparing the measured at least one of the height and the mass of the crop sample to a stored threshold; and
determining that the at least one of the height and the mass of the crop sample exceeds the stored threshold.

6. The method of claim 1, wherein the validating, comprises:
measuring an amount of produce of the crop sample;
comparing the measured amount of produce to a stored threshold; and
determining that the amount of produce of the crop sample exceeds the stored threshold.

7. The method of claim 1, wherein the validating, comprises:
determining from the crop sample that at least one biologic unit comprising the decoded DNA is alive at a current time.

8. The method of claim 1, further comprising:
selecting the at least one of the plurality of crypto farmers from the plurality of crypto farmers.

9. The method of claim 8, wherein the selecting comprises a random selection.

10. The method of claim 8, wherein the selecting is conducted based on a determination that the at least one of the plurality of crypto farmers has reached a target threshold before other crypto farmers.

11. The method of claim 1, wherein the biologic host comprises at least one of a tree, algae, bacterium, and a food crop.
